(19) United States
(12) Patent Application Publication
(10) Pub. No.: US 2010/0196929 A1
(43) Pub. Date: Aug. 5, 2010

(54) STABILISATION OF THE PHENOTYPIC PROPERTIES OF ISOLATED PRIMARY CELLS

(75) Inventors: Vera Rogiers, Bonheiden (BE); Tamara Vanhaecke, Lembeek (BE); Evelien De Rop, Lint (BE); Joanna Fraczek, Gent (BE)

Correspondence Address: KNOBBE MARTENS OLSON & BEAR LLP, 2040 MAIN STREET, FOURTEENTH FLOOR, IRVINE, CA, 92614, US

(73) Assignee: Vrije Universiteit Brussel, Brussel (BE)

(21) Appl. No.: 12/602,278

(22) PCT Filed: May 30, 2008
(86) PCT No.: PCT/EP2008/056706

§ 371 Date: Nov. 30, 2009

(30) Foreign Application Priority Data
Jun. 1, 2007 (EP) 07109456.9

(51) Int. Cl. G01N 33/567; C12N 5/071; C12P 1/00

(52) U.S. Cl. 435/7.21; 435/325; 435/41

(57) ABSTRACT

The invention provides a method for stabilisation of phenotypic properties of isolated primary cells comprising modulating at least two epigenetic modifications in said cells.

### FIELD OF THE INVENTION

The present invention relates to methods of isolation and in vitro culture of primary cells, more particularly wherein said methods aim to stabilise the phenotypic properties of the primary cells. The invention further contemplates uses of the primary cells obtainable by said methods.

### BACKGROUND OF THE INVENTION

Isolated primary cells are a desired tool, because they can closely approximate relevant facets of the in vivo situation. For instance, isolated primary cells are commonly used in the field of cell biology to analyse physiological or pathological processes that may occur in cells, tissues or organs in vivo. In another example, primary cells isolated from a patient or a suitable donor may be manipulated ex vivo, such as, e.g., transformed with a desired therapeutic gene, presented with an antigen of interest, propagated or differentiated, etc., and thereafter applied to the patient to achieve a therapeutic benefit. In a further example, isolated primary cells offer a humane alternative to the use of experimental animals in various testing and analysis applications, such as, e.g., in regulatory testing of therapeutics, cosmetics, chemicals or food additives for toxicity, carcinogenicity, catabolism or biotransformation, etc.

However, it is documented that isolated primary cells, such as, e.g., isolated primary hepatocytes, tend to gradually de-differentiate and lose their cell type- or tissue-specific properties during conventional in vitro culture. Hence, given the prevalent use of isolated primary cells as models for the respective in vivo situations, it is crucial to devise ways to isolate and culture these cells so as to better preserve their phenotypic characteristics.

WO 2006/045331, a previous application by some of the present inventors, taught that histone deacetylase (HDAC) inhibitors could stabilise the phenotypic properties of isolated primary cells, particularly of isolated primary hepatocytes. Nevertheless, in view of the demand for isolated primary cells closely representing in vivo processes, a considerable need persists in the art for additional alternatives as well as improved methods to preserve the phenotypic properties of primary cells during isolation and/or culture. Exemplary objects to be achieved in such further methods may include better or longer-term preservation of one or more relevant phenotypic properties by the isolated primary cells, and/or the use of lower amounts of agents that stabilise the phenotypic properties of the primary cells, and/or the use of shorter-term treatments to achieve the stabilisation of the phenotypic properties of the primary cells, etc.

DNA methyltransferase (DNMT) inhibitors have been shown to induce apoptosis, differentiation and/or cell cycle arrest in several cancer cell lines. In some cancer cell lines, a synergistic effect of a combination of DNMT inhibitors and HDAC inhibitors on these features has been observed (Zhu et al. 2003. Curr Med Chem Anticancer Agents 3: 187-99).

However, only scarce knowledge is available with respect to the effects of DNMT inhibitors on the phenotype of isolated primary cells, especially primary differentiated or parenchymal cells, such as for example hepatocytes.

Moreover, observations from cancer cell lines cannot be unreservedly extrapolated to healthy primary cells such as for instance to hepatocytes. To illustrate this point, exposure of primary rat hepatocytes to HDAC inhibitors produces effects different from those observed in cancer cell lines (Papeleu et al. 2005. Critical Reviews in Toxicology 35: 363-378). The most considerable discrepancy reported is seen with respect to apoptosis, since TSA, valproate and ITF2357 induce apoptosis in hepatoma cell lines, but not in primary hepatocytes.

WO2004/046312 discloses that the combination of HDAC and DNMT inhibitors achieves expansion of hematopoietic stem cells without differentiation. However, in stem cells specialised functions are as yet absent, whereas such functions need to be maintained in differentiated isolated primary cells, such as for example in hepatocytes.

### SUMMARY OF THE INVENTION

The aspects of the present invention address at least some, e.g., one or more, of the above discussed needs or objects in the art. In particular, the present inventors surprisingly found that when the phenotypic properties of isolated primary cells are preserved by modulating two or more kinds of epigenetic modifications in said cells during their isolation and/or cultivation, the phenotypic stabilisation is synergetic, i.e., the epigenetic modifications together achieve a more pronounced phenotypic stabilisation then each alone, or may even be synergistic, i.e., greater than expected for the sum of modulating each of the respective epigenetic modifications alone. This realisation may advantageously allow to obtain isolated primary cells whose one, more than one or all phenotypic properties are preserved to a greater degree and/or during a longer time period than was previously attainable. Alternatively or in addition, where the epigenetic modifications are modulated by exogenously added agents, lower quantities of such agents may be employed, thereby reducing the agents' potential toxicity to the cells or interference in downstream uses of the cells.

The present invention integrates the above realisations in its aspects. In particular, in an aspect the invention provides a method for stabilisation of phenotypic properties of isolated primary cells comprising modulating at least two epigenetic modifications in said cells. In an embodiment, the at least two epigenetic modifications are chosen from the group comprising or consisting of: acetylation of histones, methylation of histones, phosphorylation of histones, ubiquitination of histones, sumoylation of histones, ADP-ribosylation of histones and methylation of DNA.

The epigenetic modifications can be advantageously modulated by altering the expression and/or activity of cellular processes that control the respective epigenetic modifications, and more specifically by altering the expression and/or activity of cellular enzymes that catalyse or assist the addition, maintenance or removal of the (chemical) moieties constituting the respective epigenetic modifications to or from their particular target molecules, such as, e.g., histones or DNA.

In an embodiment, the methods of this aspect comprise modulating at least two epigenetic modifications in isolated primary cells, wherein one of said modulated epigenetic modifications is acetylation of histones. In another embodiment, the methods comprise modulating at least two epigenetic modifications in isolated primary cells, wherein one of said modulated epigenetic modifications is methylation of DNA. In a preferred embodiment, the methods comprise modulating at least acetylation of histones and methylation of DNA in isolated primary cells. The inventors have found that methods of this aspect which comprise modulation of acetylation of histones and/or methylation of DNA, achieve particularly pronounced stabilisation of the phenotype of isolated primary cells.

Preferably, acetylation of histones can be modulated by altering the expression and/or activity, preferably altering the activity, of one or more histone acetyltransferases (HAT), and/or altering the expression and/or activity, preferably altering the activity, of one or more histone deacetylases (HDAC), in said isolated primary cells. Preferably, methylation of DNA can be modulated by altering the expression and/or activity, preferably altering the activity, of one or more DNA methyltransferases (DNMT), and/or altering the expression and/or activity, preferably altering the activity, of one or more DNA demethylases, in said isolated primary cells.

In an embodiment, the above methods comprise—as one of said at least two modulations of epigenetic modifications—increasing the acetylation of histones in isolated primary cells. In another embodiment, the above methods comprise—as one of said at least two modulations of epigenetic modifications—reducing the methylation of DNA in isolated primary cells. In a preferred embodiment, the above methods comprise increasing the acetylation of histones and reducing the methylation of DNA in isolated primary cells. The inventors have realised that methods of the invention which comprise increasing the acetylation of histones and/or reducing the methylation of DNA, more preferably comprise both increasing the acetylation of histones and reducing the methylation of DNA, achieve particularly pronounced stabilisation of the phenotype of isolated primary cells. Preferably, acetylation of histones can be augmented by increasing the expression and/or activity, preferably increasing the activity, of one or more HAT, and/or by reducing the expression and/or activity, preferably reducing the activity, of one or more HDAC, in said isolated primary cells. Preferably, methylation of DNA can be countered or diminished by reducing the expression and/or activity, preferably reducing the activity, of one or more DNMT, and/or by increasing the expression and/or activity, preferably increasing the activity, of one or more DNA demethylases, in said isolated primary cells.

In a preferred embodiment, the above methods thus comprise—as one of said at least two modulations of epigenetic modifications—exposing the isolated primary cells to at least one HDAC inhibitor. In another preferred embodiment, the above methods comprise—as one of said at least two modulations of epigenetic modifications—exposing the isolated primary cells to at least one DNMT inhibitor. In a particularly preferred embodiment, the method for stabilisation of phenotypic properties of isolated primary cells thus comprises exposing the isolated primary cells to at least one HDAC inhibitor and at least one DNMT inhibitor.

In an embodiment, the at least two epigenetic modifications may be modulated during culturing of said isolated primary cells. In another embodiment, the at least two epigenetic modifications may be modulated during the isolation of said isolated primary cells. In a preferred embodiment, to further improve the phenotype of the isolated primary cells, the at least two epigenetic modifications can be modulated during both the isolation and subsequent culturing of said isolated primary cells. For example, during isolation, organism, organ, organ part or tissue from which the primary cells are to be isolated, may be perfused with or suspended in a solution comprising agents that can modulate said at least two epigenetic modifications. Alternatively, or preferably in addition, said agents may be added at any and preferably all subsequent steps of the employed primary cell isolation protocol.

In embodiments, primary cells may be isolated from an animal, preferably Eumetazoa, more preferably from a vertebrate, and still more preferably from a mammal, such as, preferably from a human or preferably from a non-human mammal. Primary cells may originate from or represent any organ, tissue or cell type of the organism, e.g., an animal, from which they are obtained. By means of example and without limitation, primary cells may be isolated from the central or peripheral neural system, blood, lymphatic system, blood vessels, lungs, skeletal muscle, smooth muscle, cardiac muscle, stomach, pancreas, liver, small or large intestine, connective tissue, bone, cartilage, skin, etc., of an animal.

In a preferred embodiment, primary cells are isolated from the liver of an animal, preferably a human or a non-human mammal. Particularly preferably, the primary liver cells are hepatocytes, i.e., parenchymal liver cells.

Related aspects of the invention concern the cells directly obtained and obtainable using the methods of the invention as well as uses of such cells in diagnostic, in vitro testing and therapeutic applications. The above and further aspects and preferred embodiments of the invention are described in the following sections and in the appended claims.

In a yet further aspect, the invention also provides a method for stabilisation of phenotypic properties of isolated primary hepatocytes comprising modulating methylation of DNA therein, as taught herein; as well as hepatocytes so obtained and uses of such.

### BRIEF DESCRIPTION OF FIGURES

FIG. 1 illustrates dose-dependent inhibition of DNA replication by 5-(4-dimethylaminobenzoyl)-aminovaleric acid hydroxamide (4-Me₂N-BAVAH). Hepatocytes (0.4×10⁵ cells/cm²) were cultured in duplicate and ³H-thymidine incorporation was measured at 72 hours after plating. Results shown are the means±SD from 3 independent experiments.

FIG. 2 illustrates dose-dependent inhibition of DNA replication by 5-aza-2′-deoxycytidine (decitabine, DAC). Hepatocytes (0.4×10⁵ cells/cm²) were cultured in duplicate and ³H-thymidine incorporation was measured at 72 hours after plating. Results shown are the means±SD from 6 independent experiments.

FIG. 3 illustrates dose-dependent inhibition of DNA replication by AN-8 (WO 2007/144341 and the formula as shown on page 31). Hepatocytes (0.4×10⁵ cells/cm²) were cultured in duplicate and ³H-thymidine incorporation was measured at 72 hours. Results shown are the means±SD from 6 independent experiments.

FIG. 4 illustrates dose-dependent inhibition of DNA replication by combinations of 4-Me₂N-BAVAH and DAC. Hepatocytes (0.4×10⁵ cells/cm²) were cultured in triplicate and ³H-thymidine incorporation was measured at 72 hours after plating. Results shown are the mean values and representative for the experiments performed.

FIG. 5 illustrates dose-dependent inhibition of DNA replication by combinations of 1 μM (A) and 2.5 μM (B) AN-8 and several concentrations of DAC (0-50 μM). Hepatocytes (0.4×10⁵ cells/cm²) were cultured in duplicate and ³H-thymidine incorporation was measured at 72 hours. Results shown are the means from 6 independent experiments.

FIG. 6 illustrates morphological appearance of cultured hepatocytes without EGF (−EGF), with EGF, treated (S) or not (+EGF) with 0.02% v/v ethanol. Magnification ×100 (A); morphological appearance of EGF-stimulated cultured hepatocytes treated with 5 μM AN-8 or 500 μM DAC. Magnification ×100 (B); morphological appearance of EGF-stimulated cultured hepatocytes treated with 1 μM AN-8 and/or 50 μM DAC. Magnification ×100 (C); morphological appearance of EGF-stimulated cultured hepatocytes treated with 2.5 μM AN-8 and/or 10 μM DAC. Magnification ×100 (D).

FIG. 7 illustrates albumin secretion into the culture medium of rat hepatocytes, treated or not with 500 μM DAC (A), 50 μM 4-Me₂N-BAVAH (B), or 5 μM AN-8 (C). Results are expressed as fold induction of untreated EGF-stimulated cells. Results shown are the means±SD from 4 independent experiments.

FIG. 8 illustrates albumin secretion into the culture medium of rat hepatocytes, treated or not with 25 μM 4-Me₂N-BAVAH+50 μM DAC (A); or with 1 μM AN-8 and/or 10 μM DAC (B); or with 2.5 μM AN-8 and/or 10 μM DAC (C). Results are expressed as percentage of untreated EGF-stimulated cells (n=1 for (A); for (B) and (C) results shown are the means±SD from 4 independent experiments).

FIG. 9 illustrates expression of CYP1A1 in cultured rat hepatocytes in the presence of EGF (C) and treated with several concentrations of DAC (1-500 μM) (A); AN-8 (1-5 μM) (B); 1 μM AN-8 and DAC (0-50 μM) (C); or 2.5 μM AN-8 and DAC (0-50 μM) (D). Blots shown are representative for the experiments performed.

### DETAILED DESCRIPTION OF THE INVENTION

As used herein, the singular forms “a”, “an”, and “the” include both singular and plural referents unless the context clearly dictates otherwise. By way of example, “a fibre” refers to one or more than one fibres.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within that range, as well as the recited endpoints.

The term “about” as used herein when referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, insofar such variations are appropriate to perform in the disclosed invention. However, it is to be understood that the value to which the modifier “about” refers is itself also specifically, and preferably, disclosed.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such connotation is meant to apply throughout this specification, i.e., also in the context of other aspects or embodiments, unless otherwise defined.

Hence, in an aspect, the invention provides a method for stabilisation of phenotypic properties of isolated primary cells comprising modulating at least two epigenetic modifications in said cells. Some preferred embodiments of this aspect are listed in the Summary section.

The term “isolated cell” generally denotes a cell that is not associated with one or more cells or one or more cellular components with which said cell is associated in vivo. Typically, an isolated cell may have been removed from its natural environment, e.g., from an organism, organ or tissue, or may result from propagation and/or differentiation, e.g., in vitro or ex vivo propagation and/or differentiation, of a cell that has been removed from its natural environment.

The term “primary cells” includes: cells present in a tissue, organ, or a part thereof, removed from an organism; cells present in a suspension of cells obtained from said tissue, organ, or part thereof; cells present in an explanted tissue; cells of said suspension or said explant when for the first time plated; and cells present in a suspension of cells derived from the first time plated cells.

The term “phenotype” referring to a cell denotes the whole of observable characteristics, i.e., “phenotypic properties”, of said cell. By means of example and not limitation, phenotypic properties of a cell may encompass: the morphology of the cell, such as, e.g., the shape of the cell, the dimensions or volume of the cell, the shape, size or position of the cell's nucleus, the number or size of the cell's nucleoli, etc.; the interactions of the cell with other cells or with the components of extracellular matrix; the complement of proteins, or the presence of particular protein(s), expressed by the cell; the complement of proteins, or the presence of particular protein(s), secreted by the cell; the presence of specific marker protein(s) on the cell's surface; the presence of specific enzyme(s), enzyme isotype(s), or activity of specific metabolic pathway(s) in the cell; the presence of specific signal transduction protein(s), or activity of specific signal transduction pathway(s) in the cell; the ability of the cell for contractions or movements; the ability of the cell to mediate synaptic transmission; etc.

The methods of the invention aim to stabilise at least one and preferably more than one phenotypic property of isolated primary cells. The terms “stabilise” or “stabilisation” when referring to a phenotypic property of an isolated primary cell mean that with regard to said phenotypic property the isolated primary cell remains demonstrably more similar to a cell of the corresponding cell type in vivo. For example, the methods of the invention may achieve stabilisation of at least 2, e.g., at least 3 or 4, preferably at least 5, e.g., at least 6, 7, 8 or 9, more preferably at least 10, e.g., at least 15, even more preferably at least 20, e.g., at least 50 or even at least 100 or more, distinct observable phenotypic properties of the isolated primary cells. In a preferred embodiment, the methods of the invention may thus achieve stabilisation of the overall phenotype of isolated primary cells.

For example, stabilisation of primary cells (e.g., hepatocytes) as intended herein can mean the in vitro maintenance of differentiated functions qualitatively and/or quantitatively as close as possible to their in vivo levels for a comparably longer duration than in the absence of such stabilisation.

By means of example, reference to stabilisation of phenotypic properties of primary hepatocytes may entail demonstrable preservation or maintenance of features of hepatic functionality including, without limitation, constitutive and/or inducible phase I biotransformation capacity (e.g., as demonstrated by expression and preferably activity of one or more liver-expressed CYP proteins such as inter alia members of the CYP1A, CYP2B and/or CYP3A families), expression and preferably secretion of albumin, removal of ammonia, maintenance of the cuboidal cell morphology, the formation of bile canaliculi, and others such as inter alia recited elsewhere in this specification.

The term “epigenetic modifications” refers to the various kinds of covalent modifications of chromatin, which do not change the primary nucleotide sequence of genes but nevertheless functionally modulate the phenotype of a cell, typically by regulating the pattern of gene expression, such as, e.g., by activating or suppressing the expression of particular genes or groups of genes. Epigenetic modifications can be heritable, i.e., can be transmitted to daughter cells, but are potentially reversible.

The methods of the invention modulate at least two epigenetic modifications, i.e., at least two different kinds of epigenetic modifications, in the isolated primary cell. The term “modulate” generally denotes a qualitative or quantitative alteration, including both increase and decrease, of that which is being modulated. Modulating an epigenetic modification in a cell thus refers to altering, such as increasing or decreasing, the quantity, distribution, turnover and/or inheritance of the respective covalent modification of chromatin in said cell.

The term “at least two” means 2 or more, and specifically encompasses 2, 3, 4, 5 and more than 5. Preferably, the methods of the invention may modulate between 2 and 5 epigenetic modifications, more preferably between 2 and 4 epigenetic modifications, even more preferably 2 or 3 epigenetic modifications and most preferably 2 epigenetic modifications in the isolated primary cells.

In an embodiment, the method of the invention comprises modulating at least two epigenetic modifications in isolated primary cells, wherein said at least two epigenetic modifications are chosen from the group comprising or consisting of: acetylation of histones, methylation of histones, phosphorylation of histones, ubiquitination of histones, sumoylation of histones, ADP-ribosylation of histones and methylation of DNA, preferably genomic DNA. These terms refer to the respective epigenetic modifications known as such and well-described in the art.

By means of example and without limitation, epigenetic modifications of histones may involve covalent modifications of one or more residues in one or more histone types, such as, e.g., in one or more of histones H1, H2A, H2A family members H2A.X, H2A.Z, H2A.BbD and MacroH2A.1, H₂B, H3 and H4, preferably in one or more of the core histones H2A, H₂B, H3 and H4, more preferably in one or both of histones H3 and H4. Preferably, such covalent modifications may be within the N-terminal and/or the C-terminal tail domains of said histones, more preferably at least within the N-terminal tail domain, but can also be present in the central domains of said histones.

Preferably, acetylation of a histone may involve the presence of one or more, preferably one, acetyl moiety on the ε-NH₂ group of one or more lysines and/or one or more, preferably one, acetyl moiety on the guanidino group of one or more arginines of said histone. By means of example and not limitation, in mammalian cells epigenetic acetylation of histones may preferably occur at any one, more or all of H2A histone (e.g., at K5), H₂B histone (e.g., at K5, K12, K15 and/or K20), H3 histone (e.g., at K9, K14, K18, K23 and/or K27) and/or H4 histone (e.g., at K5, K8, K12 and/or K16). By means of example and not limitation, in human cells epigenetic acetylation of histones may preferably occur at any one, more or all of H2A histone (e.g., at K5, K9, K13, K15 and/or K36), H₂B histone (e.g., at K5, K12, K15, K20, K24, K85, K108, K116 and/or K120), H3 histone (e.g., at K9, K18, K23 and/or K27) and/or H4 histone (e.g., at K5, K8, K12, K16, K20, K77, K79 and/or K91). Methylation of a histone may involve the presence of one, two or even three, methyl moieties on the ε-NH₂ group of one or more lysines and/or one or two methyl moieties on the guanidino group of one or more arginines of said histone. By means of example and not limitation, in mammalian cells epigenetic methylation of histones may preferably occur at any one, more or all of H1 histone (e.g., at K26), H3 histone (e.g., at R2, R8, K9, K14, R17, R26, K27 and/or K36) and/or H4 histone (e.g., at R3 and/or K20). By means of example and not limitation, in human cells epigenetic methylation of histones may preferably occur at any one, more or all of H1 histone (e.g., at K25), H2A histone (e.g., at K99 and/or K118), H₂B histone (e.g., at K23 and/or K43), H3 histone (e.g., at R2, K4, R8, K9, R17, K23, R26, K27, K36, K37 and/or K79) and/or H4 histone (e.g., at R3, K12, K20, K59 and/or R92). Phosphorylation of a histone may involve the presence of a phosphate moiety on the hydroxyl group of one or more serines and/or one or more threonines of said histone. By means of example and not limitation, in mammalian cells epigenetic phosphorylation of histones may preferably occur at any one, more or all of H2AX histone (e.g., at S139), H2A histone (e.g., at S1 and/or T119), H₂B histone (e.g., at S14), and/or H3 histone (e.g., at S10, T11 and/or S28). By means of example and not limitation, in human cells epigenetic phosphorylation of histones may preferably occur at any one, more or all of H1 histone (e.g., S26, T145, S171 and/or S186), H2A histone (e.g., at S1 and/or T120), H2AX histone (e.g., at T136 and/or S139), H₂B histone (e.g., at S14), H3 histone (e.g., at T3, S10 and/or S28), and/or H4 histone (e.g., at S1 and/or S47). Ubiquitination or sumoylation of a histone may involve the presence of, respectively, one or more ubiquitin and/or one or more SUMO protein on one or more lysines of said histone. By means of example and not limitation, in mammalian cells epigenetic ubiquitination of histones may preferably occur at any one, more or all of H2A histone (e.g., at K119) and/or H₂B histone (e.g., at K120). By means of example and not limitation, in human cells epigenetic ubiquitination of histones may preferably occur at any one, more or all of H2A histone (e.g., at K118) and/or H₂B histone (e.g., at K120). ADP-ribosylation of a histone may involve the presence of one (mono-ADP-ribosylation) or more (poly-ADP-ribosylation) of ADP-ribose moieties on one or more lysines, and/or one or more arginines, and/or one or more glutamates, and/or one or more dipthamide residues of said histone. The above discussed epigenetic modifications of histones are well-known to a skilled artisan and further account thereof can be found, inter alia, in Peterson & Laniel 2004 (“Histones and histone modifications”, Curr Biol 14(14): R546-R551), He & Lehming 2003 (“Global effects of histone modifications”, Brief Funct Genomic Proteomic 2(3): 234-43), Jenuwein & Allis 2001 (“Translating the histone code”, Science 293: 1074-1080), Grunstein 1997 (“Histone acetylation and chromatin structure and transcription”, Nature 389: 349-352), Kouzarides 1999 (“Histone acetylases and deacetylases in cell proliferation” Curr Opin Genet Dev 9: 40-48), Kouzarides 2002 (“Histone methylation in transcriptional control”, Curr Opin Genet Dev 12: 198-209), Gill 2004 (“SUMO and ubiquitin in the nucleus: different functions, similar mechanisms?”, Genes Dev Sep 18(17): 2046-59), and Shilatifard 2006 (“Chromatin modifications by methylation and ubiquitination: implications in the regulation of gene expression”, Annu Rev Biochem 75: 243-69).

The term “methylation of DNA” generally refers to any epigenetic modification involving the occurrence of added methyl groups in nucleotide bases of DNA, preferably of genomic DNA. In particular, the term encompasses the presence of a methyl group at the N-6 position of adenine (6-methyladenine, m⁶A), the presence of a methyl group at the N-4 position of cytosine (4-methylcytosine, m⁴C), and the presence of a methyl group at the C-5 position of cytosine (5-methylcytosine, m⁵C). Epigenetic methylation of DNA is widely addressed in the art, and detailed accounts thereof can be found in, e.g., Ng & Bird 1999 (“DNA methylation and chromatin modification”, Curr Opin Genet Devel 9: 158-163) and Robertson & Wolfe 2000 (“DNA methylation in health and disease” Nat Rev Genet. 1(1): 11-19).

The term “modulating methylation of DNA” means modulating the epigenetic methylation of DNA, and in particular modulating the methylation at any or all of the particular nucleotide bases or positions within said bases as described in the preceding paragraph. In a preferred embodiment, modulating of methylation of DNA modulates at least methylation at the C-5 position of one or more cytosines, more preferably wherein one or more or each of said cytosines forms a part of a CpG dinucleotide. In another preferred embodiment, modulating of methylation of DNA modulates only methylation at the C-5 position of one or more cytosines (i.e., does not modulate methylation at the N-6 position of adenine and the N-4 position of cytosine), more preferably wherein one or more or each of said cytosines forms a part of a CpG dinucleotide. It shall be appreciated that, without limitation, said modulation may occur globally, or may be directed towards one or more genomic regions, one or more genes or one or more sequence elements (such as, e.g., regulatory sequences, promoters, CpG islands, etc.).

In an embodiment, the methods of the invention comprise modulating at least two epigenetic modifications in isolated primary cells, wherein one of said at least two modulated epigenetic modifications is acetylation of histones. Modulating acetylation of histones may preferably increase said acetylation. By example and without limitation, the methods may modulate at least two epigenetic modifications comprising: (a) acetylation of histones and methylation of histones, or (b) acetylation of histones and phosphorylation of histones, or (c) acetylation of histones and ubiquitination of histones, or (d) acetylation of histones and sumoylation of histones, or (e) acetylation of histones and ADP-ribosylation of histones, or (f) acetylation of histones and methylation of DNA. Preferably, in any of said embodiments (a) to (f), modulating acetylation of histones may increase said acetylation.

In another embodiment, the methods of the invention comprise modulating at least two epigenetic modifications in isolated primary cells, wherein one of said at least two modulated epigenetic modifications is methylation of DNA. Modulating methylation of DNA may preferably reduce said methylation. By example and without limitation, the methods may modulate at least two epigenetic modifications comprising: (f) methylation of DNA and acetylation of histones, or (g) methylation of DNA and methylation of histones, or (h) methylation of DNA and phosphorylation of histones, or (i) methylation of DNA and ubiquitination of histones, or (j) methylation of DNA and sumoylation of histones, or (k) methylation of DNA and ADP-ribosylation of histones. Preferably, in any of said embodiments (f) to (k), modulating methylation of DNA may reduce said methylation. Preferably, said modulating, preferably reducing, methylation of DNA may modulate, preferably reduce, at least, or only, methylation at the C-5 position of one or more cytosines, more preferably wherein one or more or each of said cytosines forms a part of a CpG dinucleotide.

Particularly preferred is the above embodiment (f), wherein the method comprises modulating at least acetylation of histones and methylation of DNA in isolated primary cells. Preferably, modulating acetylation of histones in embodiment (f) may increase said acetylation. Also preferably, modulating methylation of DNA in embodiment (f) may reduce said methylation. Accordingly, in a preferred embodiment (l), the method comprises at least increasing acetylation of histones and reducing methylation of DNA in isolated primary cells. Preferably, said modulating, preferably reducing, of methylation of DNA may modulate, preferably reduce, at least, or only, methylation at the C-5 position of one or more cytosines, more preferably wherein one or more or each of said cytosines forms a part of a CpG dinucleotide.

As noted, in a preferred embodiment, an epigenetic modification can be advantageously modulated by altering the expression and/or activity of cellular processes that control said epigenetic modification, more specifically by altering the expression and/or activity of cellular enzymes that catalyse or assist the addition, maintenance or removal of the moiety constituting said epigenetic modification to or from its respective target, preferably chromatin, such as, e.g., histone(s) or DNA.

Exemplary enzymes that may regulate the epigenetic methylation of histones, and whose expression and/or activity may be altered, such as increased or decreased, to modulate said methylation, include, without limitation, histone methyltransferases, including histone-lysine N-methyltransferases, such as, e.g., SUV39H1, SUV39H2, G9A, ESET, EZH2, Eu-HMTase, SET1, MLL1, MLL4, and the like, and histone-arginine N-methyltransferases, such as, e.g., PRMT1, PRMT3, PRMT4/CARM1, PRMT5/JBP1, PRMT2, and the like (Kouzarides 2002, supra); and histone demethylases, such as, e.g., lysine-specific histone demethylase 1 (LSD1; Shi et al. 2004, “Histone demethylation mediated by the nuclear amine oxidase homolog LSD1”, Cell 119(7): 941-53) and several Jumonji (JmjC) domain-containing histone demethylation proteins, e.g., JHDM-1, 2A, 2B, 3A (see Klose et al. 2006, “JmjC-domain-containing proteins and histone demethylation”, Nat Rev Genet. 7(9): 715-27). In a preferred embodiment, the method may comprise reducing the expression and/or activity of histone methyltransferases and/or increasing the activity of histone demethylases. Exemplary enzymes that may regulate the epigenetic phosphorylation of histones, and whose expression and/or activity may be altered to modulate said phosphorylation, include, without limitation, kinases capable of phosphorylating histones, such as, e.g., Mst1 kinase (sterile twenty kinase), Msk1 kinase (mitogen- and stress-activated kinase 1), Msk2 (mitogen- and stress-activated kinase 2), Aurora B kinase, NHK-1 (nucleosomal histone kinase 1), Rsk2 kinase (ribosomal S6 kinase 2), PI3KK, Mek1 kinase (MAP kinase kinase), p38 MAP kinase, hcB kinase a (IKKα), ATR kinase (ATM and Rad3-related kinase), and the like, and phosphatases capable of de-phosphorylating histones, such as, e.g., protein phosphatase 1 (PP1), and the like. In a preferred embodiment, the method may comprise increasing the expression and/or activity of histone kinases and/or reducing the activity of histone phosphatases. Exemplary enzymes that may regulate the epigenetic ubiquitination of histones, and whose expression and/or activity may be altered to modulate said ubiquitination, include, without limitation, ubiquitin-protein ligases capable of transferring one or more ubiquitin moieties to histones, such as, e.g., Ring 1b ubiquitin ligase, the RNF 20/40 ubiquitin ligase, and the like. Exemplary enzymes that may regulate the epigenetic sumoylation of histones, and whose expression and/or activity may be altered to modulate said sumoylation, include, without limitation, SUMO-protein ligases capable of transferring one or more SUMO moieties to histones. Exemplary enzymes that may regulate the epigenetic ADP-ribosylation of histones, and whose expression and/or activity may be altered to modulate said ADP-ribosylation, include, without limitation, NAD+ADP-ribosyltransferases capable of transferring one or more ADP-ribosyl moieties to histones. See, e.g., Santos-Rosa & Caldas 2005 (“Chromatin modifier enzymes, the histone code and cancer”, Eur J Cancer 41(16): 2381-402).

As noted above, preferred methods of the invention comprise modulating, more preferably increasing, the acetylation of histones, and/or modulating, more preferably reducing, the methylation of DNA.

Exemplary enzymes that may regulate epigenetic acetylation of histones, and whose expression and/or activity may be altered to modulate said acetylation, include, without limitation, histone acetyltransferases (HAT), such as, e.g., CBP (CREB binding protein), p300, GCN5, histone acetyltransferase 1 (HAT1), P/CAF (p300/CBP associated factor), TAF1 or TAFII250 (TBP-associated factor TAFII250), ATF2 (ATF2 activating transcription factor 2), Tip60 (Tat-interacting protein 60), MYST1 histone acetyltransferase, the p160 family, including p/CIP, ACTR, TIF2/GRIP-1/NcoA-2 and SRC-1/NCoA-1, and the like; and histone deacetylases (HDAC), including class I HDAC, such as, e.g., HDAC1, 2, 3 and 8, class II HDAC including class IIa HDAC, such as, e.g., HDAC4, 5, 7 and 9, and class IIb HDAC, such as, e.g., HDAC 6 and 10, class III HDAC (sirtuins), such as, e.g., SIRT1, 2, 3, 4, 5, 6 and 7, and class IV HDAC, such as, e.g., HDAC 11. See, e.g., Kouzarides 1999 (“Histone acetylases and deacetylases in cell proliferation”, Curr Opin Genet Dev 9(1): 40-8), Hassig & Schreiber 1997 (“Nuclear histone acetylases and deacetylases and transcriptional regulation: HATs off to HDACs”, Curr Opin Chem Biol 1(3): 300-8), Gray & Ekstrom 2001 (“The human histone deacetylase family”, Exp Cell Res 262(2): 75-83) and Grozinger et al. 1999 (“Three proteins define a class of human histone deacetylases related to yeast Hdalp”, PNAS 96(9): 4868-73), for overview of HAT and HDAC, especially of mammalian or human origin.

As noted, one of said at least two epigenetic modifications modulated in the methods of the invention may preferably be acetylation of histones, and the modulation may more preferably increase said acetylation. Hence, in a preferred embodiment, the present methods may comprise increasing the expression and/or activity, preferably increasing the activity, of one or more histone acetyltransferase (HAT) in the isolated primary cells. In another preferred embodiment, the methods may comprise reducing the expression and/or activity, preferably reducing the activity, of one or more histone deacetylase (HDAC) in the isolated primary cells. For example, the methods may comprise reducing the expression and/or activity, preferably reducing the activity, of one or more class I HDAC and/or one or more class II HDAC and/or one or more class III HDAC and/or one or more class IV HDAC, in the isolated primary cells. In a further embodiment, the methods may comprise increasing the expression and/or activity, preferably increasing the activity, of one or more HAT and reducing the expression and/or activity, preferably reducing the activity, of one or more HDAC in the isolated primary cells. These actions may advantageously increase the acetylation of histones in said cells.

Exemplary enzymes that may regulate the epigenetic methylation of DNA, and whose expression and/or activity may be altered to modulate said methylation of DNA, include, without limitation, DNA methyltransferases (DNMT), such as, e.g., DNMT1, DNMT2 and DNMT3 family including DNMT3a, DNMT3b and DNMT3L, and the like; and DNA demethylases, such as, e.g., methylated DNA-binding protein 2 (MBD2/demethylase). See, e.g., Bestor 2000 (“The DNA methyltransferases of mammals”, Hum Mol Genet. 9(16): 2395-402), Siedlecki & Zielenkiewicz 2006 (“Mammalian DNA methyltransferases”, Acta Biochim Pol 53(2): 245-56) and Wolffe et al. 1999 (“DNA demethylation”, PNAS 96(11): 5894-6) for discussion of DNMT and DNA demethylases, especially of mammalian or human origin.

As noted, one of said at least two epigenetic modifications modulated in the methods of the invention may preferably be methylation of DNA, and the modulating may more preferably decrease said methylation of DNA. Hence, in a preferred embodiment, the present methods may comprise reducing the expression and/or activity, preferably reducing the activity, of one or more DNA methyltransferases (DNMT) in the isolated primary cells. For example, the methods may comprise reducing the expression and/or activity, preferably reducing the activity, of DNMT1 and/or DNMT2 and/or one or more members of the DNMT3 family, in the isolated primary cells. In another preferred embodiment, the methods may comprise increasing the expression and/or activity, preferably increasing the activity, of one or more DNA demethylases in the isolated primary cells. In a further embodiment, the methods may comprise reducing the expression and/or activity, preferably reducing the activity, of one or more DNMT and increasing the expression and/or activity, preferably increasing the activity, of one or more DNA demethylases in the isolated primary cells. These actions may advantageously increase the methylation of DNA in said cells.

As noted, the at least two epigenetic modifications modulated in the methods of the invention may preferably comprise acetylation of histones and methylation of DNA. More preferably, the method may comprise increasing acetylation of histones and decreasing methylation of DNA. Hence, in various preferred embodiments the method of the invention may comprise: (1) and (3); or (1) and (4); or (2) and (3); or (2) and (4); or (1) and (2) and (3); or (1) and (2) and (4); or (1) and (3) and (4); or (2) and (3) and (4); or (1) and (2) and (3) and (4); wherein “(1)” means increasing the expression and/or activity, preferably increasing the activity, of one or more HAT in the isolated primary cells; “(2)” represents reducing the expression and/or activity, preferably reducing the activity, of one or more HDAC (such as, e.g., of one or more class I HDAC and/or one or more class II HDAC and/or one or more class III HDAC and/or one or more class IV HDAC) in the isolated primary cells; “(3)” means reducing the expression and/or activity, preferably reducing the activity, of one or more DNMT (such as, e.g., of DNMT1 and/or DNMT2 and/or any or all DNMT3 members) in the isolated primary cells; and “(4)” represents increasing the expression and/or activity, preferably increasing the activity, of one or more DNA demethylase in the isolated primary cells. In a particularly preferred embodiment, the method comprises (2) and (3) as defined herein, providing for particularly advantageous stabilisation of phenotypic properties of the isolated primary cells.

Hence, the methods of the invention typically comprise altering the expression and/or activity of one or more proteins, e.g., of enzymes as exemplified above.

The term “altering expression of a protein” means effecting a change, including both increase and decrease, in the level of expression of said protein in a cell, particularly in the isolated primary cells. The term encompasses any extent of change.

Preferably, where expression level of a protein in a cell is increased, the increase may be by at least about 10%, e.g., at least about 20%, at least about 30% or at least about 40%, preferably by at least about 50%, e.g., at least 60% or at least 70%, more preferably by at least about 70%, e.g., at least about 80% or at least about 90%, even more preferably by at least about 100%, e.g., by at least about 150%, still more preferably by at least about 200%, e.g., by at least about 300%, at least about 400% or even by at least about 500% or more, relative to the basal expression level of to the protein in the cell (i.e., the expression level in the cell without effecting said increase). Preferably, where expression level of a protein in a cell is decreased, the decrease may be by at least about 10%, e.g., at least about 20%, more preferably by at least about 30%, e.g., at least about 40%, yet more preferably by at least about 50%, e.g., at least about 60%, still more preferably by at least about 70%, e.g., at least about 80%, and most preferably by at least about 90%, e.g., at least about 95% or even about 100%, relative to the basal expression level of the protein in the cell. The expression levels of proteins may be determined and compared using quantification methods routinely known in the art, such as, for example, ELISA, RIA, immuno-precipitation, Western blotting, etc.

An increase in the expression level of a protein in a cell may be achieved by methods known in the art, such as, e.g., by transfecting (e.g., by electroporation, lipofection, etc.) or transducing (e.g., using a viral vector), the cell with a recombinant nucleic acid which encodes said protein under the control of a promoter effecting suitable expression level in said cell. A decrease in the expression level of a protein in a cell may be achieved by methods known in the art, such as, e.g., by transfecting (e.g., by electroporation, lipofection, etc.) or transducing (e.g., using a viral vector), the cell with an antisense agent, such as, e.g., antisense DNA or RNA oligonucleotide, a construct encoding for an antisense transcript, or an RNAi agent, such as siRNA, shRNA, etc. Alternatively, an increase or decrease in the expression level of a protein might be achieved using suitable chemical or biological activators or inhibitors, respectively.

The term “altering activity of a protein” means effecting a change, including both increase and decrease, in the amount of activity of said protein in a cell, particularly in the isolated primary cells. The term “activity” particularly relates to the biochemical or enzymatic activity of the protein, and more specifically means that activity which affects the epigenetic modifications in the cell. The term encompasses any extent of change in activity.

Preferably, where activity of a protein in a cell is increased, the increase may be by at least about 10%, e.g., at least about 20%, at least about 30% or at least about 40%, preferably by at least about 50%, e.g., at least 60% or at least 70%, more preferably by at least about 70%, e.g., at least about 80% or at least about 90%, even more preferably by at least about 100%, e.g., by at least about 150%, still more preferably by at least about 200%, e.g., by at least about 300%, at least about 400% or even by at least about 500% or more, relative to the basal amount of activity of the protein in the cell (i.e., the amount of activity in the cell without effecting said increase). Preferably, where activity of a protein in a cell is decreased, the decrease may be by at least about 10%, e.g., at least about 20%, more preferably by at least about 30%, e.g., at least about 40%, yet more preferably by at least about 50%, e.g., at least about 60%, still more preferably by at least about 70%, e.g., at least about 80%, and most preferably by at least about 90%, e.g., at least about 95% or even about 100%, relative to the basal amount of activity of the protein in the cell. The activity of proteins may be determined and compared using quantification methods routinely known in the art, e.g., in vitro methylation/demethylation assays, acetylation/deacetylation assays, phosphorylation/deposphorylation assays, etc. of the respective substrates. For example, various enzymatic assays exist in the art to assess histone acetylation activity, histone methylation activity, histone phosphorylation activity, DNA methylation activity, etc. An increase or decrease in the activity of a protein in a cell may be advantageously achieved using suitable chemical or biological activators or inhibitors (e.g., chemical substances, intrabodies, dominant negative forms, etc.) of said protein, respectively.

Acetylation of histones may be preferably modulated, in particular increased, by exposing cells to one or more HDAC inhibitors. Accordingly, in an embodiment the method for stabilisation of phenotypic properties of isolated primary cells comprises exposing said cells to at least one HDAC inhibitor. Methylation of DNA may be preferably modulated, in particular reduced, by exposing cells to one or more DNMT inhibitors. Accordingly, in an embodiment the method for stabilisation of phenotypic properties of isolated primary cells comprises exposing said cells to at least one DNMT inhibitor. In a particularly preferred embodiment, the invention thus provides a method for stabilisation of phenotypic properties of isolated primary cells comprising exposing said cells to at least one HDAC inhibitor and at least one DNMT inhibitor.

The term “expose” or “exposing” means bringing cells in contact with the agent to which they are said to be exposed. Preferably, said exposure may be achieved by including said agent in one or more reagents used for isolating the cells and/or one or more culture media used to culture said cells.

The term “histone deacetylase inhibitor” or “HDAC inhibitor” denotes an agent capable of decreasing the activity (as defined above) of at least one histone deacetylase in cells. In exemplary, non-limiting embodiments, an HDAC inhibitor may inhibit: one or more class I HDAC; or one or more class II HDAC; or one or more class III HDAC; or one or more class IV HDAC; or one or more class I HDAC and one or more class II HDAC; or all class I HDAC; or all class II HDAC; or all class III HDAC; or all class IV HDAC; or all class I HDAC and one or more class II HDAC; or one or more class I HDAC and all class II HDAC; or all class I HDAC and all class II HDAC; etc. Preferably, an HDAC inhibitor may inhibit at least one or more class I HDAC, such as, e.g., all class I HDAC, and/or at least one or more class II HDAC, such as, e.g., all class II HDAC.

HDAC inhibitors may take the form of a chemical or biological substance, a pharmaceutical agent or drug, a therapeutically effective oligonucleotide, a specific binding agent, or a fragment or variant of histone deacetylase. For example, an HDAC inhibitor may be a small organic molecule inhibitor, preferably having size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The HDAC inhibitor may preferably be a specific HDAC inhibitor, i.e., an agent that, while inhibiting the activity of one or more HDAC, does not substantially directly affect other enzyme systems, and wherein the effect(s) on downstream cellular components and functions are a direct consequence of that agent.

Several structural classes of histone deacetylase inhibitors have been identified and may be used in the invention, comprising naturally occurring as well as synthetic compounds. These include, by means of example and not limitation, benzamides (e.g., MS-27-275, CI-994), sulfonamide-based anilides, straight chain anilides, heterocyclic ketones (e.g., apicidin and its derivatives, FR235222), allyl sulfur compounds (e.g., diallyl sulfide), psammaplins (e.g., psammaplin A), heterocyclic thiols (e.g., depsipeptide, spiruchostatin A), sulfur-containing cyclic peptides, bromoacetamide-based straight chain inhibitors, short-chain fatty acids (e.g., sodium butyrate), pivaloyloxymethyl butyrate, tributyrin, phenylalkanoic acids, valproic acid, phenylbutyrate, N-acetylcystein sulforaphane, cystein sulforaphane, straight chain trifluoromethylketones, alpha-ketoamides, alpha-ketoesters, heterocyclic ketones, cyclic trifluoromethylketones, cyclic pentafluoromethylketones, heterocyclic epoxides (e.g., trapoxin A), depudecin, short-chain fatty hydroxamic acids, trichostatins (e.g., trichostatin A), hybrid polar compound (e.g., suberoyl anilide hydroxamic acid, pyroxamide), benzamide-based hydroxamic acids (e.g., 6-(4-dimethylaminobenzoyl)aminocaproic acid hydroxamide, 5-(4-dimethylaminobenzoyl)-aminovaleric acid hydroxamid), arylketones, amino acid-containing benzamide based hydroxamic acids, indole amide-based hydroxamic acids, aryl-heterocyclic-based hydroxamic acids, aryl-pentadienoic hydroxamic acids, sulfonamide-based hydroxamic acids (e.g., oxamflatin, PXD101), (bi-)aryl-(heterocyclic)-based hydroxamic acids (e.g., SK-7041, SK-7068), NVP-LAQ824, A-161906, LBH589, S379872-A, aroyl-pyrrole-hydroxyamides, cyclic hydroxamic acid-containing peptides, cyclic hexapeptides, succinimide hydroxamic acids, cystein-based hydroxamic acids, 1.3-dioxanes (e.g., tubacin), phosphorus-based straight chain inhibitors, cyclostellettamines (e.g., cyclic 3-alkyl pyridinium dimers), straight-chain retrohydroxamates, cyclic retrohydroxamates, semicarbazide-based straight chain inhibitors, and thiol-based straight chain inhibitors. See, e.g., Bolden 2006 (“Anticancer activities of histone deacetylase inhibitors”, Nat Rev Drug Discov 5(9): 769-84). Accordingly, in an embodiment of the present invention, the one or more HDAC inhibitors may be chosen from HDAC inhibitors falling into one of the above structural classes, such as, e.g., one of the above individualised HDAC inhibitors. However, it will be appreciated that all presently known HDAC inhibitors, whether indicated above or not, may be used in the method according to the present invention. Also, novel HDAC inhibitors identified in the future can be used in the method according to the present invention. In addition, any HDAC inhibitor may be used as a derivative, e.g., as a biologically acceptable salt, e.g., in order to exert maximum effect in cells.

In a preferred embodiment, the method may employ Trichostatin A (TSA), which is a potent specific HDAC inhibitor, in particular of Class I and Class II HDAC (Yoshida et al. 1990. J Biol Chem 265:17174-17179, 1990). Trichostatin A may be brought in the form of derivates with HDAC inhibitory activity, such as salts, preferably biologically acceptable salts, which may be used in the present method.

In another embodiment, semi-synthetic or synthetic TSA analogues, or derivatives thereof with HDAC inhibitory activity, such as salts, and preferably biologically acceptable salts, may be employed. For example, Jung et al. 1999 (J Med Chem 42: 4669-4679) described the production and properties of several amide-based TSA analogues, including, e.g., 6-(4-dimethylaminobenzoyl)-aminocaproic acid hydroxamid. For example, Van Ommeslaeghe et al. 2003 (Bioorg Med Chem Lett 13:1861-1864) described the production of several amide-based TSA analogues, among others 5-(4-dimethylaminobenzoyl)-aminovaleric acid hydroxamid (4-Me₂N-BAVAH). In another example, 5-(4-dimethylaminobenzoyl)-aminovaleric acid hydroxamid is a potent HDAC inhibitor and may be metabolically more stable than TSA.

The term “DNA methyltransferase inhibitor” or “DNMT inhibitor” denotes an agent capable of decreasing the activity (as defined above) of at least one DNA methyltransferase in cells. In exemplary, non-limiting embodiments, an HDAC inhibitor may inhibit: at least DNMT 1; or at least DNMT 2; or at least one of DNMT 3 family, preferably at least DNMT 3a and/or DNMT 3b; or at least DNMT1 and DNMT2; or at least DNMT1 and at least one of DNMT3 family, preferably at least DNMT 3a and/or DNMT 3b; or at least DNMT 2 and at least one of DNMT3 family, preferably at least DNMT 3a and/or DNMT 3b; or at least DNMT1 and DNMT2 and at least one of DNMT3 family, preferably at least DNMT 3a and/or DNMT 3b.

DNMT inhibitors may take the form of a chemical or biological substance, a pharmaceutical agent or drug, a therapeutically effective oligonucleotide, a specific binding agent, or a fragment or variant of DNMT. For example, an DNMT inhibitor may be a small organic molecule inhibitor, preferably as described above. The DNMT inhibitor may preferably be a specific DNMT inhibitor, i.e., an agent that, while inhibiting the activity of one or more DNMT, does not substantially directly affect other enzyme systems, and wherein the effect(s) on downstream cellular components and functions are a direct consequence of that agent.

Several structural classes of DNMT inhibitors have been identified and may be used in the invention, comprising naturally occurring as well as synthetic compounds. These include, by means of example and not limitation, nucleoside analogue DNMT inhibitors (e.g., 5-azacytidine, 5-aza-2′-deoxycytidine or decitabine, 1-β-D-arabinofuranosyl-5-azacytosine or fazarabine, dihydro-5-azacytidine or DHAC, and zebularine), non-nucleoside analogue DNMT inhibitors (e.g., procaine, procainamide, (−)epigallocatechin-3-gallate, psammaplins), and oligonucleotide substrates (e.g., MG98). See, e.g., Lyko & Brown 2005 (“DNA methyltransferase inhibitors and the development of epigenetic cancer therapies”, J Natl Cancer Inst 97(20): 1498-506) for exemplary description of DNMT inhibitors. Accordingly, in an embodiment of the present invention, the one or more DNMT inhibitors may be chosen from DNMT inhibitors falling into one of the above structural classes, such as, e.g., one of the above individualised DNMT inhibitors. However, it will be appreciated that all presently known DNMT inhibitors, whether indicated above or not, may be used in the method according to the present invention. Also, novel DNMT inhibitors identified in the future can be used in the method according to the present invention. In addition, any DNMT inhibitor may be used as a derivative, e.g., as a biologically acceptable salt, e.g., in order to exert maximum effect in cells.

In a preferred embodiment, the method may employ decitabine which may optionally be brought in the form of derivates with DNMT inhibitory activity, such as salts, preferably biologically acceptable salts, which may be used in the present method.

As noted, in an embodiment, the at least two epigenetic modifications may be modulated in the primary cells (preferably, the primary cells may be exposed to agents modulating said at least two epigenetic modifications; such as, e.g., modulating acetylation of histones and/or methylation of DNA; such as, e.g., exposure to one or more HDAC inhibitors and/or one or more DNMT inhibitors) during culturing of said cells, i.e., during in vitro (ex vivo) tissue culturing of said cells. Preferably, said modulating may commence upon or after the time (t=0) of first plating of the primary cells following isolation. Preferably, said modulating may commence no later than at t=48 h, more preferably no later than at t=24 h, even more preferably no later than at t=12 h, still more preferably no later than at t=6 h, yet more preferably no later than at t=4 h, e.g., no later than at t=3 h, t=2 h, t=1 h or t=0.5 h, and very preferably said modulating may commence at t=0 h.

Also preferably, said modulating may be sustained throughout a substantial part of the total duration of in vitro culturing of said primary cells, such as throughout at least 50% of the total duration, preferably throughout at least 60%, more preferably throughout at least 70%, even more preferably throughout at least 80%, still more preferably throughout at least 90%, and most preferably throughout about 100% of the total duration of in vitro culturing of said primary cells.

In another preferred embodiment, at least two epigenetic modifications may be modulated in the primary cells before the first plating of the primary cells (t<0), i.e., during isolation of said cells. In a further embodiment, the at least two epigenetic modifications may be modulated in the primary cells both during isolation (t<0) and during culturing (t≧0) of said cells. Preferably, organism, organ, organ part or tissue from which the primary cells are to be isolated, may be perfused with or suspended or submerged in a composition, preferably liquid composition, configured to effect said modulating, e.g., comprising suitable agents as described elsewhere in this specification. The term “perfuse” refers to introducing the composition into an organism, organ, organ part or tissue through blood vessels supplying said. Alternatively, or preferably in addition, said modulating is also effected at any and preferably all subsequent steps of the primary cell isolation protocol.

It shall be appreciated that agents used to modulate the at least two epigenetic modifications during isolation of the primary cells (t<0) may be the same as or different from the agents used during culturing of said cells (t≧0). Also, it shall be appreciated that agents used to modulate the at least two epigenetic modifications may be the same or may vary throughout said culturing. It shall also be appreciated that the at least two epigenetic modifications that are modulated during isolation of the primary cells (t<0) may be the same as or may be different from the at least two epigenetic modifications modulated during culturing of said cells (t≧0). Moreover, it shall be appreciated that the modulated at least two epigenetic modifications may be the same or may vary throughout said culturing.

As noted, the primary cells may be isolated from an animal, preferably Eumetazoa, more preferably from a vertebrate, and still more preferably from a mammal, such as, preferably from a human or preferably from a non-human mammal. The term “mammal” includes any animal classified as such, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes. Primary cells may be isolated using established methods from organs, organ parts or tissues obtained from living or deceased animals using techniques available in the art, such as, e.g., biopsy, dissection, resection, etc.

Primary cells according to the invention may originate from or represent any organ, tissue or cell type of the organism, e.g., an animal, from which they are obtained. By means of example and without limitation, primary cells may be isolated from the central or peripheral neural system, blood, lymphatic system, blood vessels, lungs, skeletal muscle, smooth muscle, cardiac muscle, stomach, pancreas, liver, small or large intestine, connective tissue, bone, cartilage, skin, etc., of an animal.

In a preferred embodiment, the primary cells may be differentiated, preferably terminally differentiated, i.e., fully specialised cells that take up specialised functions in various tissues and organs of an organism, and which may but need not be post-mitotic. In another embodiment, the primary cells may be parenchymal cells, i.e., cells performing or contributing to organ-specific functions.

In a preferred embodiment, primary cells as intended herein do not encompass stem cells, such as embryonic, foetal or adult stem cells, such as inter alia haematopoietic stem cells.

Hence, in a preferred but non-limiting embodiment, the isolated primary cells may be chosen from the group comprising or consisting of the following cell types: neurons (e.g., CNS or PNS neurons), neuroglia (e.g., astrocytes, Schwan cells, oligodendrocytes), myocytes, cardiomyocytes, pneumocytes, pancreatic beta-cells, hepatocytes, endothelial cells, epithelial lining cells, keratinocytes, osteocytes, osteoclasts, chondrocytes, adipocytes and leukocytes.

It shall be appreciated that the primary cells may be isolated from a healthy subject or from a subject suffering a disease. For example, cells isolated from a healthy subject may allow to study the normal physiology of the isolated cells and/or to use said cells in applications requiring normal functions from such cells. In another preferred embodiment, the cells may be isolated from a subject suffering a disease. Furthermore, said disease may, directly or indirectly, affect the organ, tissue or cell type from which said primary cells are isolated. Advantageously, this may inter alia allow to analyse the pathology in the isolated cells (even on a patient-customised basis).

In a particularly preferred embodiment, the primary cells are isolated from the liver of an animal. More preferably, the primary cells are hepatocytes, for which the inventors realised particular effectiveness of the method of the invention. The term “hepatocyte” encompasses epithelial, parenchymal liver cells, including but not limited to hepatocytes of different sizes (e.g., “small”, “medium-size” and “large” hepatocytes), ploidy (e.g., diploid, tetraploid, octaploid) or other characteristics. For example, some authors propose that “large” hepatocytes, as defined therein, are the parenchymal cells responsible for physiological functions of the liver, and accordingly, the primary cells may be preferably “large” hepatocytes.

Hepatocytes may be isolated from liver using cell disassociation and isolation methods well known in the art. For example, isolation of liver cells from liver tissue has been well known since the mid-1960s (Howard et al. 1967, J Cell Biol 35: 675-84). Rat hepatocytes were isolated using a combined mechanical and enzymatic digestion technique, subsequently modified by Berry and Friend 1969 (J Cell Biol 43: 506-20). This technique was further developed by Seglen to become the widely used two-step collagenase perfusion technique (Methods Cell Biol 13: 29-83, 1976). A skilled person is aware that since the above publication of the said technique, various modifications thereof have been described and/or are conceivable, and are included in the invention. Hence, in a particularly preferred embodiment, hepatocytes may be isolated as detailed in Papeleu et al. 2006 (Methods Mol Biol 320: 229-37). In a further instance, methods employing cell sorting and FACS analysis may be used.

A skilled person is aware that two-step collagenase techniques may be particularly suited to release hepatocytes from liver tissue. Cell suspensions obtained using the said technique may comprise a considerable proportion of primary hepatocytes.

Accordingly, in a particularly preferred embodiment, the invention provides a method for stabilisation of phenotypic properties of isolated primary hepatocytes, comprising modulating at least two epigenetic modifications in said hepatocytes. As noted above, in one of particularly preferred embodiments, said method may comprise modulating, preferably increasing, acetylation of histones and/or (preferably “and”) modulating, preferably reducing, methylation of DNA in said hepatocytes. In a further one of particularly preferred embodiments, the method may comprise reducing the expression and/or activity of one or more HDAC and/or (preferably “and”) reducing the expression and/or activity of one or more DNMT in said hepatocytes. Hence, in a particularly preferred embodiment, the method may comprise exposing said hepatocytes, during culturing, and preferably also in the course of isolation, to one or more HDAC inhibitors and/or (preferably “and”) to one or more DNMT inhibitors.

The present inventors further identified that cultured hepatocytes may predominantly express class I HDAC 1, 2, 3 and 8, class IIa HDAC 7 and class IV HDAC 11, while remaining HDAC may not be expressed or may be expressed at relatively low levels. Accordingly, in isolated primary hepatocytes the method of the invention may preferably target (e.g., modulate expression and/or activity, preferably reduce the expression and/or activity, such as, e.g., using HDAC inhibitors) any or all of the above HDAC. For example, all above HDAC may be targeted using broad spectrum HDAC inhibitors, such as, e.g., TSA, SAHA, PXD101, LBH589 or LAQ824. In another embodiment, class I and IIa HDAC may be specifically inhibited by, e.g., valproic acid, sodium butyrate or trapoxin. Alternatively, specific ones of the above HDAC may be inhibited, such as HDAC 1 and 2 (e.g., using depsipeptide, SK-7041 or SK-7068), HDAC 1, 2 and 3 (e.g., using MS-275) or HDAC 8 (e.g., using SB-379872), whereby specific inhibition profiles may be attained.

The present inventors also found that cultured hepatocytes may predominantly express DNMT 2 and DNMT 3a, while remaining DNMT may not be expressed or may be expressed at relatively low levels. Accordingly, in isolated primary hepatocytes the method of the invention may preferably target (e.g., modulate expression and/or activity, preferably reduce the expression and/or activity, such as, e.g., using DNMT inhibitors) DNMT 2 and/or DNMT 3a.

A skilled person is generally knowledgeable about the phenotypic characteristics of hepatocytes and can thus assess the stabilisation of one or more of such phenotypic features by the methods of the present invention. By means of further illustration but not limitation, such hepatocyte phenotypic features stabilised by the methods of the invention may involve any one or more or all of: —polygonal to cubical morphology; —binucleation or higher ploidy; —expression of one or more liver enriched transcription factors, such as, e.g., HNF-3β, HNF-1α, HNF-4-α and/or C/EPBα; —expression of functional liver proteins, such as, e.g., albumin, anti-trypsin and/or transthyretin; —expression and/or activity of phase I biotransformation enzymes, such as, e.g., CYP1A (e.g., CYP1A1), CYP2B and/or CYP3A cytochrome P450 family of enzymes; —expression and/or activity of phase II biotransformation enzyme GST; —expression and/or activity of cytochrome P450; —expression of cytokeratin CK18; —expression of connexin Cx32; etc.

In a further aspect, the invention provides isolated primary cells, particularly isolated primary hepatocytes, obtainable or directly obtained using the methods of the invention.

The invention also provides a composition, preferably pharmaceutical composition, comprising isolated primary cells, particularly isolated primary hepatocytes, obtainable or directly obtained using the methods of the invention.

Furthermore, the invention contemplates the use of isolated primary hepatocytes obtainable or directly obtained using the methods of the invention, for the manufacture of a medicament for the treatment of liver disease. Also provided is a method for the manufacture of a medicament for the treatment of liver disease comprising: —stabilising the phenotypic properties of isolated differentiated primary hepatocytes as taught herein, and using said cells for manufacturing the medicament for the treatment of liver diseases. Accordingly, the invention also provides a method for treating a liver disease in a patient in need of such treatment, comprising administering a therapeutically effective amount (i.e., an amount sufficient to elicit a desired local or systemic effect) of isolated primary hepatocytes obtainable or directly obtained using the methods of the invention to said patient. For instance, said isolated primary hepatocytes can be transplanted or injected to a patient. For example, said isolated primary hepatocytes could be of therapeutic benefit in the treatment of liver based inborn, metabolic deficiencies. Non exhaustive examples of such diseases include phenylketonuria and other aminoacidopathies, haemophilia and other clotting factor deficiencies, familial hypercholesterolemia and other lipid metabolism disorders, urea cycle disorders, glycogenosis, galactosemia, fructosemia, tyrosinemia, protein and carbohydrate metabolism deficiencies, organic aciduria, mitochondrial diseases, peroxysomal and lysosomal disorders, protein synthesis abnormalities, defects of liver cell transporters, defect of glycosylation and the like. Other disease states or deficiencies typified by loss of liver mass and/or function, that could benefit from the isolated primary hepatocytes of the invention, include but are not limited to Alagille syndrome, alcoholic liver disease (alcohol-induced cirrhosis), a1-antitrypsin deficiency (all phenotypes), hyperlipidemias and other lipid metabolism disorders, autoimmune hepatitis, Budd-Chiari syndrome, biliary atresia, progressive familial cholestasis type I, II and III, cancer of the liver, Caroli Disease, Crigler-Najjar syndrome, fructosemia, galactosemia, carbohydrate deficient glycosylation defects, other carbohydrate metabolism disorders, Refsum disease and other peroxysomal diseases, Niemann Pick disease, Wolman disease and other lysosomal disorders, tyrosinemia, triple H, and other amino acid metabolic disorders, Dubin-Johnson syndrome, fatty liver (non alcoholi steato hepatitis), Gilbert Syndrome, Glycogen Storage Disease I and III, hemochromatosis, hepatitis A-G, porphyria, primary biliary cirrhosis, sclerosing cholangitis, tyrosinemia, clotting factor deficiencies, hemophilia B, phenylketonuria, Wilson's Disease, fulminant liver failure, post hepatectomy liver failure, mitochondrial respiratory chain diseases. In addition, the cells can also be used to treat acquired liver disorders due to viral infections, such as, e.g., hepatitis, e.g., type A, B, C, D or E.

In another aspect, the invention provides use of isolated primary cells, particularly isolated primary hepatocytes, obtainable or directly obtained by the methods of the invention in assays of toxicity.

Hence, also provided is an assay of toxicity comprising: —stabilising the phenotypic properties of isolated differentiated primary hepatocytes as taught in any of claims 1 to 9, and—assaying toxicity in said cells. Many candidate therapeutics are significantly hepatotoxic. Therefore, in drug development, great significance is attached to the potential toxicity a candidate therapeutic may have to hepatocytes. Such in vitro assays examine the toxicity to cultured cells or suspended cells of compounds or compositions, e.g., chemical, pharmaceutical, cosmetic or biological compounds or compositions, or biological agents. In this context, a particular compound or composition may be considered toxic or likely toxic, if it shows a detrimental effect on the viability of cells or on one or more aspect of cellular metabolism or function. Typically, the viability of cells in vitro may be measured using colorimetric assays, such as, e.g., the MTT (or MTT derivative) assays or LDH leakage assays, or using fluorescence-based assays, such as, e.g., the Live/Dead assay, CyQuant cell proliferation assay, or assays of apoptosis. Other assays may measure particular aspects of cellular metabolism or function.

In another aspect, the invention provides use of isolated primary cells, in particular isolated primary hepatocytes, obtainable or directly obtained by the methods of the invention in assays of carcinogenicity. Hence, also provided is an assay of carcinogenicity comprising: —stabilising the phenotypic properties of isolated differentiated primary hepatocytes as taught in any of claims 1 to 9, and—assaying carcinogenicity in said cells. Such in vitro assays examine the carcinogenicity, including both genotoxic and non-genotoxic (i.e., epigenetic) carcinogenicity, to cultured cells or suspended cells of compounds or compositions, e.g., chemical, pharmaceutical, cosmetic or biological compounds or compositions, or biological agents. In this context, a particular compound or composition may be considered carcinogenic or likely carcinogenic, if it induces neoplastic transformation of the cells, or induces phenotypic changes in the cells that may be predictive of such neoplastic transformation, or induces genetic or metabolic changes that may potentially cause such neoplastic transformation. Such phenotypic changes in the cells may, by means of example but not limitation, comprise morphological transformation, increased proliferation, dedifferentiation, independence of attachment, removal of contact inhibition of cells grown in monolayers, or expression of specific marker proteins. Such genetic changes in the cells may, by means of example but not limitation, comprise DNA damage, chromosomal aberrations, such as chromosomal rearrangements, alterations in chromosome number (aneuploidy), or karyotype aberrations, gene mutations, such as point mutations, deletions or insertions. Compounds or compositions that cause this kind of genetic changes are often referred to as mutagenic or mutagens.

In another aspect, the invention provides use of isolated primary hepatocytes obtainable or directly obtained by the methods of the invention in assays of biotransformation. Hence, also provided is an assay of biotransformation comprising: —stabilising the phenotypic properties of isolated differentiated primary hepatocytes as taught herein, and—assaying biotransformation by said cells. The pharmacokinetics of a candidate therapeutic belongs to factors determining its usefulness. For example, low metabolic stability may result in poor availability and high clearance of such candidate therapeutic. Moreover, while in most cases biotransformation of drugs results in bio-inactivation, it is also possible that pharmacologically active, reactive, or toxic intermediates and metabolites are produced. Therefore, the biotransformation of a new chemical entity, such as a drug or a candidate therapeutic, needs to be investigated as early in the drug discovery process as possible. The liver represents the major site of xenobiotic biotransformation and/or detoxification of a wide variety of foreign compounds, including many therapeutic agents, and the pharmacokinetics of a candidate therapeutic are heavily influenced by their biotransformation in the liver. Hepatocyte cultures thus represent one in vitro system capable of integrated biotransformation, improved by the present invention. Hence, such cultures may provide indication of the biotransformation of, e.g., chemical, pharmaceutical or biological compounds or compositions, as the outcomes of such assays may prove highly predictive of the biotransformation of the respective various compounds or compositions by primary hepatocytes in vivo and/or in vitro. Within relation to biotransformation assays, the isolated primary hepatocytes of the invention such differentiated cell will ideally have a biotransformation capacity, i.e., either phase I or phase II, or both phase I and phase II biotransformation capacities, sufficiently comparable to the hepatocytes in vivo or to freshly isolated primary hepatocytes.

In another aspect, the invention provides use of isolated primary cells, preferably isolated primary hepatocytes, obtainable or directly obtained by the methods of the invention for preparation of a biochemical extract there from. Such extract may primarily contain, by means of example but not limitation, the cytosolic fraction, the nuclear fraction, the cytoplasmic membrane fraction, the nuclear membrane fraction, the microsomal fraction, the mitochondrial fraction, the endoplasmic reticuluum fraction, the Golgi apparatus fraction, the lysosomal fraction, the cytoskeletal fraction, etc. A preferred application for the extract from isolated primary hepatocytes may be its use in in vitro biotransformation assays.

Preferably, said extract may be microsomal extract, preferably from isolated primary hepatocytes according to the invention. Microsomal extracts or suspensions will contain the smooth endoplasmic reticulum of the differentiated cells and may be used in biotransformation assays to evaluate, for example, phase I oxidation reactions, cytochrome P450 (CYP)-dependent inhibitory drug-drug interactions and the importance of genetic polymorphisms in biotransformation.

In a further aspect, the invention provides the use of isolated primary hepatocytes obtainable or directly obtained using the methods of the invention for the manufacture of an artificial liver device. Such devices, for example the Extracorporeal Liver Assist Device (ELAD) will be well-known to the person skilled in the art.

In a further aspect, the methods of the invention may use hepatocytes from diseased liver (e.g., from a subject suffering from a liver disease as specified above), which may be employed inter alia to study the pathogenic mechanisms of the respective diseases.

The above aspects and embodiments are further supported by the following examples which are in no instance to be considered limiting.

### EXAMPLES

### Example 1

### Anti-Proliferative Activity of 4-Me₂N-BAVAH or AN-8 and/or Decitabine in EGF-Stimulated Rat Hepatocytes

In the present study we investigated the effects of the DNMT inhibitor decitabine (DAC) and/or two HDAC inhibitors, namely 4-Me₂N-BAVAH and AN-8 (see WO 2007/144341 and the formula below) on proliferation of primary hepatocytes.

The following formula shows the structure of AN-8:

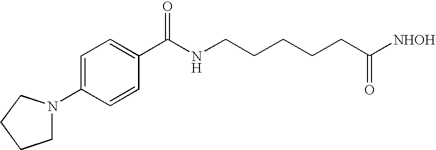

### Materials and Methods

### Cell Cultures:

Hepatocytes (viability >80%) were isolated from male Sprague-Dawley rats by a two-step collagenase perfusion. Cells (0.4×10⁵ cells/cm²) were cultured in MEM/M199 (3:1, v/v), containing 1 mg/ml BSA, 5 μg/ml bovine insulin, 2 mM L-glutamine, antibiotics (7.3 IU/ml benzyl penicillin, 50 μg/ml streptomycin sulphate, 50 μg/ml kanamycin monosulphate and 10 μg/ml sodium ampicillin) and 10% (v/v) fetal calf serum.

After 4 h, medium was removed and serum-free medium, containing 0.5 μg/ml hydrocortisone and 50 μg/ml EGF, was added and renewed daily.

DAC was dissolved in PBS, 4-Me₂N-BAVAH and AN-8 were dissolved in ethanol, to a concentration of 100 mM for DAC and 4-Me₂N-BAVAH and 25 mM for AN-8. Final ethanol concentration in media did not exceed 0.05% v/v and did not affect proliferative activity.

Three culture conditions were used:

1) unstimulated cultures, referred to as “negative control” or “−EGF”, where hepatocytes are arrested at the mitogen-dependent R-point.
2) EGF-stimulated cultures, referred to as “positive control” or “+EGF”, in which EGF induces G1/S transition and mitosis.
3) EGF-stimulated cultures treated with DAC and/or in combination with 4-Me₂N-BAVAH or AN-8.

### DNA Synthesis:

DNA replication was measured as incorporation of radioactively labeled thymidine into newly synthesized DNA. Cells were incubated with [methyl-3H]-thymidine (25 Ci/mmol) for 24 hours and harvested at 72 hours in ice-cold PBS, the time point where DNA synthesis in untreated EGF-stimulated cultures is maximal. DNA was precipitated with 30% trichloroacetic acid (TCA) overnight at 4° C. After centrifugation, cells were washed once with 10% TCA and twice with 5% TCA. The pellet was solved in formic acid and radioactively labeled thymidine was measured by liquid scintillation counting.

### Cell Morphology:

Cell morphology was analyzed by inverse light microscopy, using an Optiphot Nikon Phase Contrast microscope.

### Results

4-Me₂N-BAVAH AN-8 and DAC Inhibit DNA Synthesis in EGF-Stimulated Hepatocytes

From the moment of seeding, adult primary rat hepatocytes, stimulated with EGF 4 hours after plating and constantly thereafter, were exposed to different concentrations of 4-Me₂N-BAVAH (10 μM, 15 μM, 20 μM, 25 μM and 50 μM), AN-8 (1 μM, 2.5 μM and 5 μM) or DAC (1 μM, 10 μM, 50 μM, 100 μM, 250 μM and 500 μM). [methyl-3H]-Thymidine incorporation was measured at 72 hours.

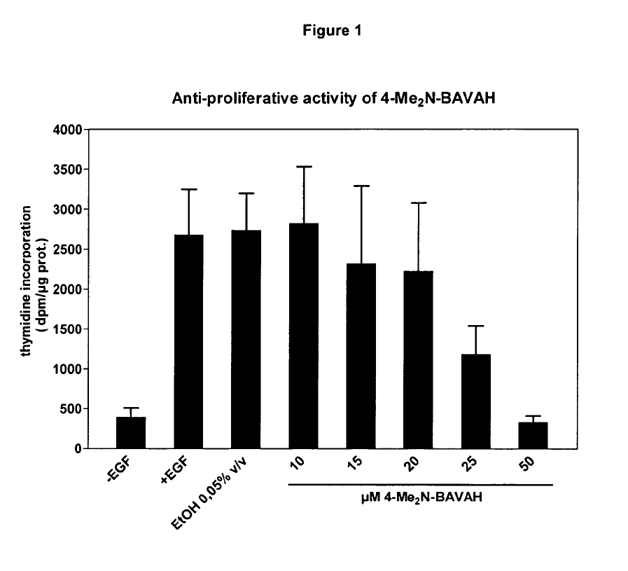

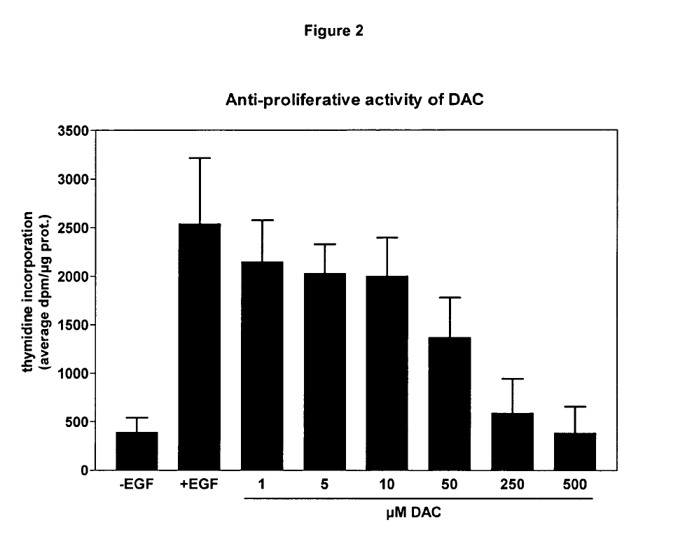

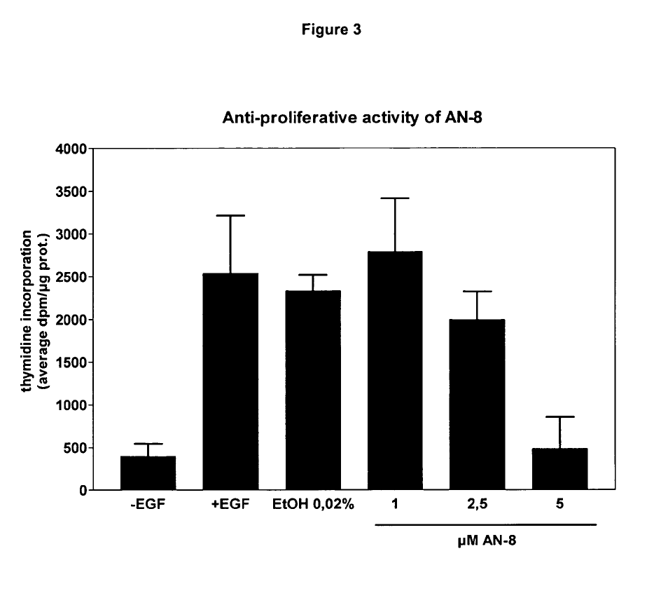

Treatment of EGF-stimulated hepatocytes with increasing concentrations of 4-Me₂N-BAVAH, AN-8 and DAC, revealed a clear dose-response relationship (FIGS. 1, 2 and 3). In absence of EGF, no DNA replication occurred.

50 μM 4-Me₂N-BAVAH, 5 μM AN-8 and 500 μM DAC caused complete inhibition of the DNA synthesis.

4-Me₂N-BAVAH and AN-8 inhibit DNA synthesis in EGF-stimulated hepatocytes at lower concentrations when combined with decitabine

From the moment of seeding, adult primary rat hepatocytes, stimulated with EGF 4 hours after plating and constantly thereafter, were exposed to different combinations of 4-Me₂N-BAVAH and DAC or AN-8 and DAC:

$$\begin{matrix}10 μM 4 - {Me}_{2} N - BAVAH \\ 15 μM 4 - {Me}_{2} N - BAVAH \\ 20 μM 4 - {Me}_{2} N - BAVAH \\ 25 μM 4 - {Me}_{2} N - BAVAH\end{matrix} \} + \begin{matrix}1 μM DAC \\ 5 μM DAC \\ 10 μM DAC \\ 50 μM DAC\end{matrix} \\ \begin{matrix}1 μM AN - 8 \\ 2 , 5 μM AN - 8\end{matrix} \} + \begin{matrix}1 μM DAC \\ 5 μM DAC \\ 10 μM DAC \\ 50 μM DAC\end{matrix}$$

[methyl-3H]-Thymidine incorporation was measured at 72 hours.

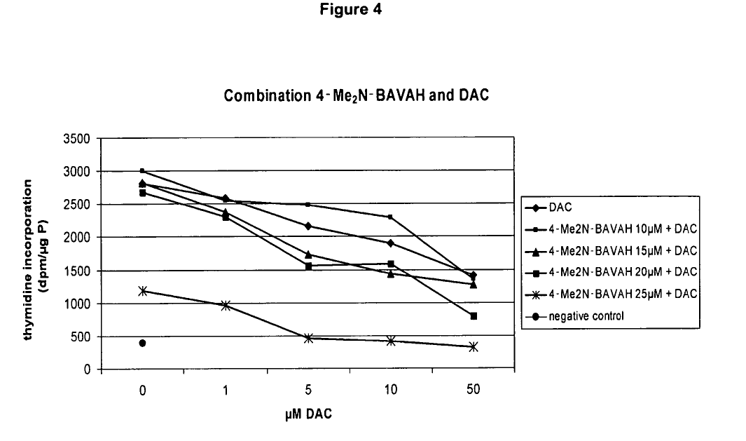

Treatment of EGF-stimulated hepatocytes with different combinations of 4-Me₂N-BAVAH and DAC, shows that 4-Me₂N-BAVAH, in combination with decitabine, is inhibiting the DNA replication at lower concentrations (FIG. 4).

For example, 25 μM 4-Me₂N-BAVAH alone causes 64.7-24.8% inhibition of DNA replication; whereas the same concentration of 4-Me₂N-BAVAH, combined with 10 μM DAC, causes an 99.1% inhibition.

These results show that HDAC inhibitors and DNMT inhibitors have a synergetic or even synergistic effect and that both mechanisms are important to maintain differentiation by preventing proliferation in primary adult rat hepatocytes in vitro.

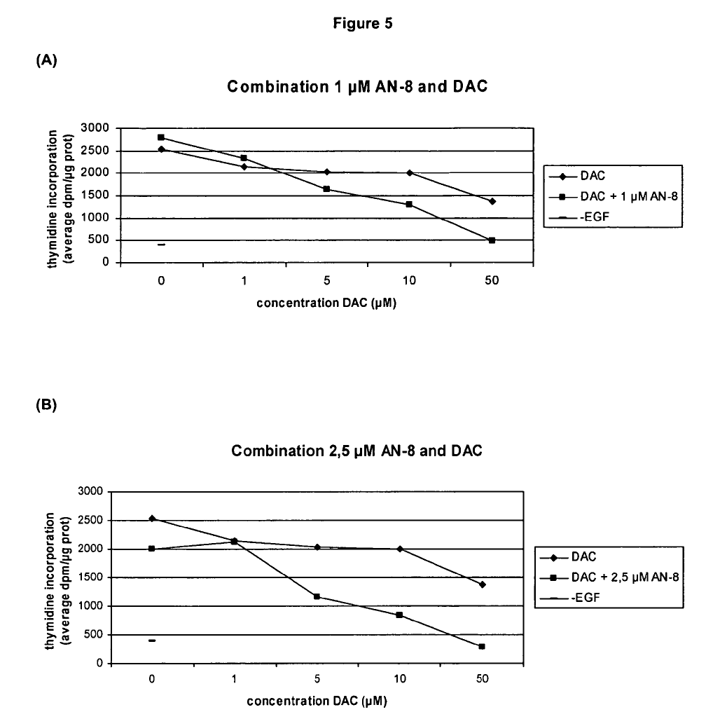

Also the treatment of EGF-stimulated hepatocytes with different combinations of AN-8 and DAC was investigated (FIGS. 5A and B).

In comparison to the single treatment of EGF-stimulated primary rat hepatocytes with either AN-8 or DAC, we observed that complete inhibition of DNA synthesis was reached by using much lower concentrations of AN-8 and DAC.

Morphological Appearance of EGF-Stimulated Hepatocytes Treated with AN-8 and/or DAC.

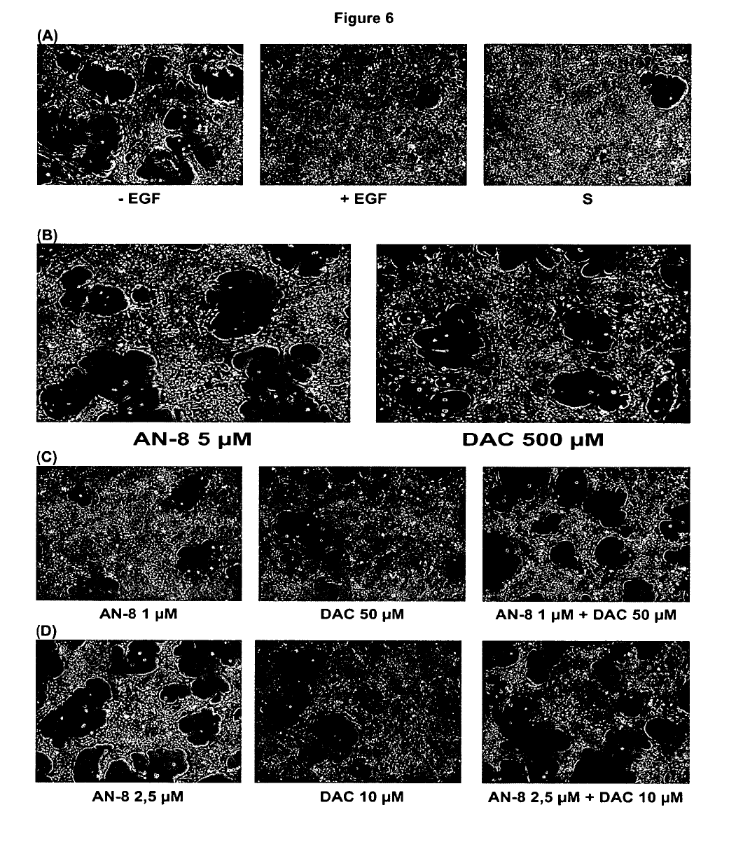

Morphological examination of the three control hepatocyte cultures showed low proliferation for EGF-deprived cultures and nice proliferation for EGF-containing cultures treated or not with the vehicle ethanol (FIG. 6A). These photos, in line with the results from the DNA synthesis, demonstrate again that ethanol has no effect on the proliferative activity of cultured hepatocytes.

In addition to the anti-proliferative effects seen by the analysis of DNA synthesis, photos of cultured EGF-stimulated hepatocytes treated with either 5 μM AN-8 or 500 μM DAC showed a clear inhibition of proliferation in comparison with the images of the untreated EGF-stimulated hepatocyte culture (+EGF) (FIG. 6B).

Also the morphological appearance of cultured hepatocytes, treated with 5 μM AN-8 or 500 μM DAC, retained better the in vivo-like cuboidal shape when compared to untreated control cultures in the presence of EGF alone.

In comparison to the single treatment of EGF-stimulated primary rat hepatocytes, we also observed by morphological examination that inhibition of proliferation was more pronounced when using combinations of AN-8 and DAC and this at lower concentrations for both compounds (FIGS. 6C and 6D).

### Example 2

### Effects of 4-Me₂N-BAVAH, AN-8 and/or Decitabine on Hepatocyte Function

In the present study the secretion of albumin, a general parameter of hepatocyte differentiation, was first investigated.

### Materials and Methods

### Cell Cultures:

Hepatocytes (viability >80%) are isolated from male Sprague-Dawley rats by a two-step collagenase perfusion. Cells (0.4×10⁵ cells/cm²) are cultured in MEM/M199 (3:1, v/v), containing 1 mg/ml BSA, 5 μg/ml bovine insulin, 2 mM L-glutamine, antibiotics (7.3 IU/ml benzyl penicillin, 50 μg/ml streptomycin sulphate, 50 μg/ml kanamycin monosulphate and 10 μg/ml sodium ampicillin) and 10% (v/v) fetal calf serum.

After 4 h, medium was removed and serum-free medium, containing 0.5 μg/ml hydrocortisone and 50 μg/ml EGF, is added and renewed daily.

DAC and 4-Me₂N-BAVAH were dissolved as above.

Two culture conditions were used:

1) EGF-stimulated cultures, referred to as “positive control” or “control”.
2) EGF-stimulated cultures treated with DAC and/or in combination with 4-Me₂N-BAVAH or AN-8.

### Albumine Secretion:

Medium samples were analyzed for their albumin content by an enzyme-linked immunosorbent assay (ELISA) according to Dunn et al. 1991 (Biotechnol Prog 7(3): 237-245).

### Results

### 4-Me₂N-BAVAH, AN-8 and DAC Increase Albumin Secretion in EGF-Stimulated Hepatocytes

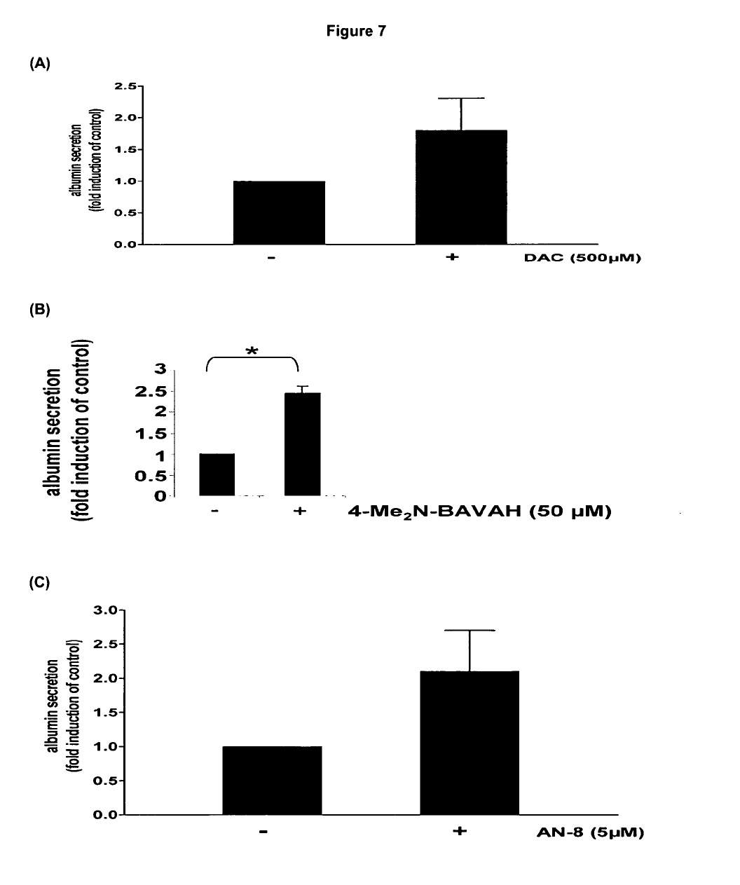

From the moment of seeding, adult primary rat hepatocytes, stimulated with EGF 4 hours after plating and constantly thereafter, were exposed to 50 μM 4-Me₂N-BAVAH, 5 μM AN-8 or 500 μM DAC. Albumin secretion into the culture medium was measured between 48-72 hours of culture (FIG. 7A-C).

Compared to the control cultures, a 1.8, 2.4 and 2.1 fold increase in albumin secretion was observed in the presence of 500 μM DAC, 50 μM 4-Me₂N-BAVAH and 5 μM AN-8, respectively.

Increased Albumin Secretion in EGF-Stimulated Hepatocytes when 4-Me₂N-BAVAH or AN-8 are Combined with Decitabine

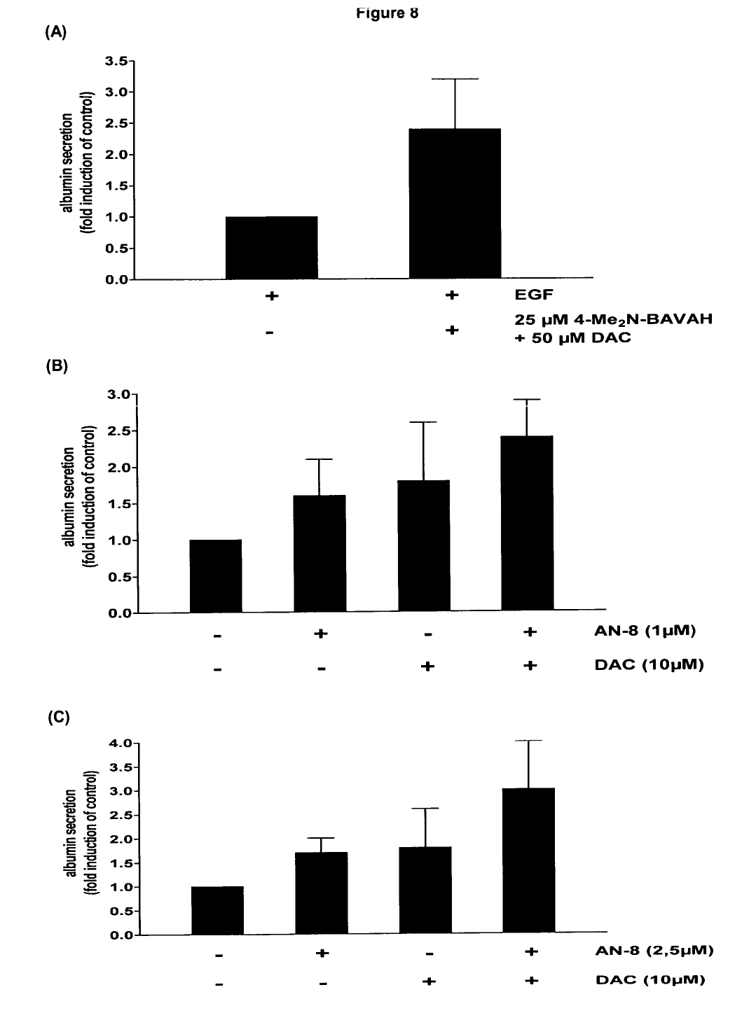

From the moment of seeding, adult primary rat hepatocytes, stimulated with EGF 4 hours after plating and constantly thereafter, were exposed to a combination of 25 μM 4-Me₂N-BAVAH and 50 μM DAC. Albumin secretion into the culture medium is measured between 48-72 hours of culture (FIG. 8A).

Treatment of EGF-stimulated hepatocytes with a combination of 4-Me₂N-BAVAH and DAC, shows an increased albumin secretion already at lower concentrations of both 4-Me₂N-BAVAH and DAC, in the combination.

In addition, in the presence of several combinations of DAC and AN-8, an overall higher albumin secretion was observed in comparison to the single treatment of EGF-stimulated primary rat hepatocytes to either of the compounds (FIGS. 8B and C).

### Example 3

### Effects of AN-8 and/or Decitabine on CYP1a1 Protein Expression in EGF-Stimulated Primary Rat Hepatocytes

Next to the beneficial effects of the combinations of DNMT inhibitors and/or HDAC inhibitors on the inhibition of hepatocyte proliferation and the albumin secretion in EGF-stimulated primary rat hepatocytes, we next wanted to know if these inhibitors also positively affect the protein expression of CYP1A1, an important phase I xenobiotic biotranformation isoenzyme.

### Materials and Methods

### Cell Cultures:

Hepatocytes (viability >80%) were isolated from male Sprague-Dawley rats by a two step collagenase perfusion. Cells (0.4×10⁵ cells/cm²) were cultured in MEM/M199 (3:1, v/v), containing 1 mg/ml BSA, 5 μg/ml bovine insulin, 2 mM L-glutamine, antibiotics (7.3 IU/ml benzyl penicillin, 50 μg/ml streptomycin sulphate, 50 μg/ml kanamycin monosulphate and 10 μg/ml sodium ampicillin) and 10% (v/v) fetal calf serum. After 4 h, medium was removed and serum-free medium, containing 0.5 μg/ml hydrocortisone and 50 μg/ml EGF, was added and renewed daily. DAC was dissolved in PBS, whereas AN-8 was dissolved in ethanol to a concentration of 100 mM for DAC and 25 mM for AN-8. Final ethanol concentration in media did not exceed 0.02% v/v.

Three culture conditions were used:

1) EGF-stimulated cultures, referred to as C.
2) EGF-stimulated cultures, exposed to 0.02% v/v ethanol, referred to as S.
3) EGF-stimulated cultures treated with DAC and/or AN-8.

### Immunoblot Analysis:

At 72 h, cultured hepatocytes were harvested in ice-cold PBS. Cell pellets were lysed in homogenization buffer, sonicated and left for 1 hour on ice. After centrifugation, the protein concentration in the supernatant was determined. 50 μg proteins were separated by sodium dodecyl sulphate polyacrylamide gel electrophoresis (SDS-PAGE, 7.5%) and transferred to nitrocellulose membranes. Subsequently, membranes are immersed into Ponceau S to examine equal sample loading. Further, membranes were blocked for 1 h at room temperature with 5% (w/v) non-fat milk in Tris buffered saline to avoid non-specific binding of the antibodies. Incubation with primary antibody was performed overnight under gentle shaking at 4° C., followed by incubation with HRP-labeled secondary antibody for 1 h at room temperature. Proteins were detected by using an enhanced chemiluminescence detection system.

### Results

Elevated CYP1A1 Expression in EGF-Stimulated Hepatocytes after Exposure to DAC and AN-8

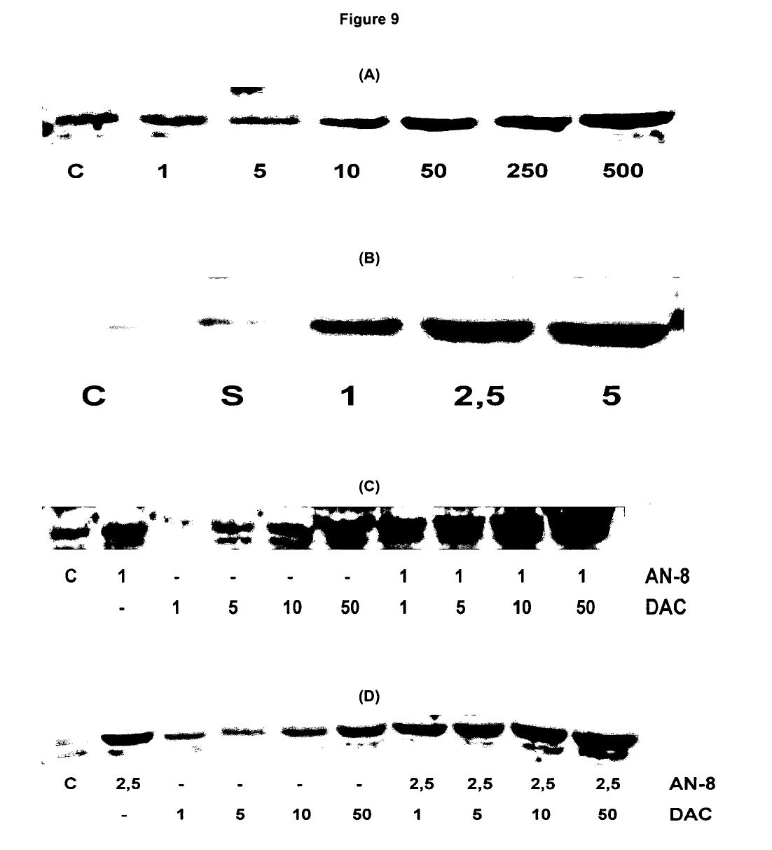

From the moment of seeding, adult primary rat hepatocytes, stimulated with EGF 4 hours after plating and constantly thereafter, were exposed to different concentrations of either DAC (1 μM, 10 μM, 50 μM, 250 μM and 500 μM) or AN-8 (1 μM, 2.5 μM and 5 μM). Treatment of EGF-stimulated hepatocytes with increasing concentrations of DAC and AN-8, showed a dose-dependent higher CYP1A1 protein expression in comparison to untreated hepatocyte cultures (FIGS. 9A and B).

In addition, in the presence of several combinations of DAC and AN-8, an overall higher CYP1A1 expression level was observed when compared to the single treatment of EGF-stimulated primary rat hepatocytes with these compounds, even using lower concentrations (FIGS. 9C and D).

These results indicate that HDAC inhibitors and DNMT inhibitors in combination have a synergetic or even synergistic effect and that both mechanisms are important to maintain a differentiated status in primary adult rat hepatocytes in vitro, through prevention of proliferation.

Indeed, upon exposure of the cells to HDAC inhibitors, DNMT inhibitors or their combination, the primary cells better maintain their functionality as shown by an importantly increased expression of CYP1A1 in comparison with appropriate controls (FIG. 9). This is notable, in particular because cells were cultured under so-called ‘proliferation conditions’, meaning in the presence of EGF and a low cell density. Under these conditions, primary cells are normally forced to proceed through the cell cycle, thereby mutually excluding differentiation (Loyer et al. 1996. J Biol Chem 271: 11484-11492; Loyer et al. 1996. Progress in Cell Cycle Research 2: 37-47). Without exposure to HDAC inhibitors, DNMT inhibitors or their combination, the cells in culture (in particular rat hepatocytes) have significantly decreased albumin secretion capacity, loose their 3-dimensional shape and nearly completely loose their in vivo CYP activity and expression already within 24 hours remaining at that very low level during further culture. This is also observed here for CYP1A1 under control conditions as described in example 3 (FIG. 9 control condition C, 72 hours of culture). However, with exposure to HDAC inhibitors, DNMT inhibitors and particularly their combination, high CYP1A1 expression levels and thus a higher differentiation degree are observed throughout culture, as shown here after 72 hours under proliferation conditions.

1. A method for stabilisation of phenotypic properties of isolated differentiated primary cells, comprising modulating at least two epigenetic modifications in said isolated differentiated primary cells.

2. The method according to claim 1, wherein the differentiated primary cells are terminally differentiated cells.

3. The method according to claim 1, wherein the differentiated primary cells are parenchymal cells.

4. The method according to claim 1, wherein the differentiated primary cells are hepatocytes.

5. The method according to claim 1, wherein the at least two epigenetic modifications are selected from the group consisting of: acetylation of histones, methylation of histones, phosphorylation of histones, ubiquitination of histones, sumoylation of histones, ADP-ribosylation of histones, and methylation of DNA.

6. The method according to claim 5, comprising modulating at least acetylation of histones and methylation of DNA in said isolated differentiated primary cells.

7. The method according to claim 6, wherein said modulating of acetylation of histones increases said acetylation, and wherein said modulating of methylation of DNA reduces said methylation.

8. The method according claim 1, comprising exposing the isolated differentiated primary cells to at least one HDAC inhibitor and at least one DNMT inhibitor.

9. The method according to claim 1, wherein said at least two epigenetic modifications are modulated during isolation and/or culturing of the isolated differentiated primary cells.

10. An assay of biotransformation comprising:
stabilising the phenotypic properties of isolated differentiated primary hepatocytes as taught in claim 1, and
assaying biotransformation by said cells.

11. An assay of toxicity or carcinogenicity comprising:
stabilising the phenotypic properties of isolated differentiated primary hepatocytes as taught in claim 1, and
assaying toxicity or carcinogenicity in said cells.

12. A method for the manufacture of a medicament for the treatment of liver disease comprising:
stabilising the phenotypic properties of isolated differentiated primary hepatocytes as taught in claim 1, and
manufacturing the medicament for the treatment of liver diseases using said cells.

13. The method of claim 7, wherein increasing said acetylation comprises increasing the expression and/or activity of one or more histone acetyltransferases (HAT) and/or by reducing the expression and/or activity of one or more histone deacetylases (HDAC).

14. The method of claim 7, wherein reducing said methylation comprises reducing the expression and/or activity of one or more DNA methyltransferases (DNMT) and/or by increasing the expression and/or activity of one or more DNA demethylases.
